(19) United States
(12) Patent Application Publication
(10) Pub. No.: US 2005/0130325 A1
(43) Pub. Date: Jun. 16, 2005

(54) Fluorescent beads detecting method and fluorescent beads detecting apparatus

(75) Inventors: Yoshitada Oshida, Kanagawa (JP); Hiroshi Kishida, Tokyo (JP)

Correspondence Address: Reed Smith Hazel & Thomas LLP;Suite 1400, 3110 Fairview Park Drive, Falls Church, VA, 22042-4503, US

(73) Assignee: Hitachi Software Engineering Co., Ltd.

(21) Appl. No.: 11/006,624

(22) Filed: Dec. 8, 2004

(30) Foreign Application Priority Data
Dec. 12, 2003 (JP) 2003-415376

(51) Int. Cl. C12M 1/34; G01J 3/42; G01J 3/427; G01N 33/544; G01N 33/546

(52) U.S. Cl. 436/528; 435/287.2; 356/319

(57) ABSTRACT

A method and a device for accurately detecting with fluorescence some arranged beads through utilization of fluorescent beads in a flow path because each diameter the beads is small in respect to an inner diameter of the flow path and the beads are not arranged in a linear manner. A fine elongated excitation light is radiated in a direction perpendicular to the flow path to the samples having a plurality of units with fluorescent members being applied to some relative large beads, this is relatively scanned in a direction toward the flow path so as to perform a well-separable and accurate detection of the beads without being influenced by the adjoining beads.

### FIELD OF THE INVENTION

The present invention relates to a method and an apparatus for detecting fluorescence emitted from a sample having a plurality of beads arranged in a flow path to include fluorescent substance on or in the beads.

### BACKGROUND OF THE INVENTION

As a useful and efficient method for checking a state of appearance of genes, there has been started to use DNA probe arrays or DNA chips having many DNA probes classified by their types and fixed on a surface of a solid member. In JP-A. No. 243997/1999 described below, there has been disclosed an invention about a probe array in which some particles (probe particles) having various probes fixed thereto and arrayed under a specified order. More practically, a plurality of fine tubes or grooves filled with each of the probe particles are arranged side by side, each of the particles is poured into another fine tube or groove one by one from each of the fine tubes or grooves so as to make the probe array having various probe particles always arranged under a specified order, the probe arrays are used and several kinds of probes are connected to the particles having different particle diameters through the probe arrays and then several kinds of fluorescent marker DNAs are concurrently measured.

In the technology described in the patent document 1 below, it is possible to fix the bio-molecules to be analyzed to the surfaces of the beads and analyze them by a method wherein the capillary beads array in which some beads with various kinds of probe particles being fixed to their surfaces are arranged in a flow path such as a glass capillary under a desired order is manufactured, sample solution containing bio-molecules to be analyzed is fed to the capillary beads array, the bio-molecules to be analyzed are fixed to the surfaces of the beads and the bio-molecules are coupled to the probe molecules having a high affinity.

When the bio-molecules with the beads are analyzed, fluorescent substance is added to some unknown targets such as DNA, protein and immunity, substances having already-known substances composed of one type to a plurality of types of substances adhered to the surfaces of the beads are put into a flow path having a larger inner diameter than the diameter of the beads under a predetermined order. Although a certain combination of the already-known substances at the surfaces of the beads and the un-known substances added with fluorescent substance shows that they are coupled to each other, another combination except the former one shows that they are not coupled to each other. Accordingly, if the beads radiating the excitation light and emitting the fluorescent light are acknowledged, a substance to be inspected is acknowledged. When the substance in which the un-known object to be inspected having the aforesaid fluorescent substance added thereto is dissolved in liquid such as water is flowed into the flow path, this solution flows fast in a narrow space between the beads and the flow path wall, resulting in that the aforesaid coupling reaction is produced rapidly and so a problem found in this type of inspection that the reaction time is long can be solved.

As described above, although it is advantageous for the method for using the fluorescent beads to promote the reaction, the beads are not arranged in a linear form because the beads in the flow path have a smaller diameter as compared with an inner diameter of the flow path. Due to this fact, it was difficult to perform an accurate detecting operation when the arranged beads are detected through fluorescence. That is, if a large diameter spot beam is radiated against the beads for radiating the excitation light positively against the fluorescent beads according to the prior art method, the excitation light is also radiated against the adjoining beads and a discrete detection for every beads is not performed well. In addition, in turn when a small spot beam was radiated inversely against the beads, this showed a problem that certain beads were radiated with beam against a high position near the central portions and other certain beads were radiated against a surrounding deep position and a quantitative detection could not be attained.

### SUMMARY OF THE INVENTION

In order to solve the aforesaid prior art problem found in detecting the fluorescent beads, the present invention provides the following means.

First, the present invention is an invention of a method for detecting fluorescent beads and a shape of excitation light to be radiated against the fluorescent beads is set to such a shape as one having a different horizontal to vertical ratio. A side of this shape in its longitudinal direction is directed perpendicular to that of a flow having a large flow path as compared with that of the short side and then a position on the beam on which the excitation light radiates is changed in sequence in a relative manner. Fluorescence generated through radiation of this excitation light is detected while being separate from the excitation light so as to attain a desired fluorescent signal. In this case, it is preferable that a length of the excitation light in a longitudinal direction is smaller than a diameter of sectional area perpendicular to a flowing direction of the flow path and larger than a value by times of 0.3 of the beads diameter. With this arrangement as above, it becomes possible to radiate the excitation light against the fluorescent beads more positively without radiating the excitation light against the adjoining beads.

Further, it is preferable that either the aforesaid excitation light or a position detecting beam having a specified positional relation with the aforesaid excitation light guides a light reflected at the beads to a light position detector, determines a timing for detecting fluorescence based on the position signal detected by the light position detector to cause the fluorescence to be detected. With such an arrangement as above, it becomes possible to detect fluorescence while taking a time in which the excitation light is radiated onto a top part of each of the beads to enable the fluorescence to be detected and further an accurate, i.e. a quantitative fluorescence detection to be carried out.

Further, when the fluorescence generated through radiation of the excitation light is separately detected from the excitation light, it is preferable to use at least one excitation light-fluorescence separation means from among three units of a wavelength selection beam splitter, a spatial filter and an interference filter. In order to determine whether any one type, any two types, or all the types of the means are used, the means is selected according to a precision to be required or its cost.

In order to detect accurately a time in which the excitation light radiates against the top parts of the beads, it is necessary for only the reflected light from the beads to be caught. Due to this fact, it is preferable that as either the excitation light or the position detecting beam, a light linearly polarized through a polarizer used at a light source for a linear polarized light or a light source for non-polarized light is applied through the polarized light beam splitter and the wavelength plate. That is, the excitation light passes through P-polarization at the polarization beam splitter in a going light path directing toward the beads, becomes an S-polarized light through an intermediate wavelength plate, in particular, ¼ wavelength plate at a returning path where the light reflects against the beads and returns, the light reflects at the polarized beam splitter and incident to the light position detector. With such an arrangement as above, all the lights becoming noise component reflected at the surface of the optical system placed between the polarized beam splitter and the wavelength plate pass through the polarized beam splitter, do not reach to the light position detector, resulting in that a positional detection with a less amount of noise can be carried out. In this case, the lowest noise can be attained if the aforesaid ¼ wavelength plate occupies a location between an objective lens and the beads.

Employing the aforesaid method enables a quite high precise fluorescent detection to be carried out. However, in order to restrict the noise more and perform a high precise detection, it is preferable to arrange the fluorescent beads including the aforesaid fluorescent at its surface or inside it and dummy beads not including the fluorescent substance at its surface or inside it or fluorescent beads including different kinds of fluorescent substances under a specified order. If such a sample is used, it becomes possible to remove almost of all noises generated from the adjoining beads.

Secondly, the present invention is an invention of fluorescent beads detecting apparatus applied in the aforesaid fluorescent beads detecting method for radiating an excitation light against beads including at surfaces or inside part thereof fluorescent substances arranged in a flow path and detecting the fluorescent emitted from the beads comprising an exciting light source; a beam forming optical system for forming light beams emitted from the light source in a shape having a different horizontal to vertical ratio on the fluorescent beads and with a long direction of the shape being directed toward a direction perpendicular to a large flow in the flow path as compared with that of the short direction; an excitation light-fluorescent branch means for separating the excitation light from the fluorescence; a wavelength selection beam splitter for either reflecting or passing light depending on a difference in wavelength between the excitation light and the fluorescence; a radiation optical system for radiating (excitation light) the excitation light passed through the beam splitter against the beads including at the surface or inner part the fluorescent substance acting as detected item; a detecting optical system for detecting the fluorescence emitted from the fluorescent substance; a fluorescent detector for detecting fluorescence passed through the detecting optical system; a position detector to which an excitation light reflected from the aforesaid beads or light emitted from the light source separately arranged from the aforesaid excitation light is radiated so as to detect the light reflected from the aforesaid beads and passed through the aforesaid excitation light beam splitter; a fluorescent detector; and a control circuit for attaining information of the fluorescent beads based on information got from the position detector and the fluorescent detector.

It is preferable that the fluorescence beads detecting apparatus of the present invention is set such that the aforesaid lighting optical system and the detecting optical system have a common objective lens.

In addition, it is preferable that the aforesaid beam splitter is a polarized beam splitter in the same manner as that of the first invention and a ¼ wavelength plate is installed between the aforesaid objective lens and the beads.

In addition, as the aforesaid excitation light-fluorescent branch means, it is preferable to select and use at least one type of excitation light-fluorescent separating means in reference to the excitation light-fluorescent separating means from among three units of wavelength selection beam splitter, spatial filter and interference filter.

Further, it is preferable that the fluorescent detector device of the present invention is provided with a driving means for changing a relative position between the aforesaid fluorescent beads and the excitation light.

The present invention has enabled a sample having fluorescence applied to the beads to be easily and accurately detected with fluorescence and further enabled a convenient and less-expensive inspection to be carried out.

### BRIEF DESCRIPTION OF THE DRAWINGS

FIG. 1 shows a preferred embodiment of the present invention;

FIG. 2 shows an item to be detected when the beads are detected according to the present invention;

FIG. 3 shows a detail of an item to be detected when the beads are detected according to the present invention;

FIG. 4 shows a preferred embodiment of a beam forming optical system;

FIG. 5 is an illustration for showing a position sensor used for performing beads detection;

FIG. 6 is an illustration for showing a detecting signal for use in detecting beads detection according to the present invention;

FIG. 7 shows another preferred embodiment of the present invention;

FIG. 8 shows a still another preferred embodiment of the present invention; and

FIG. 9 shows a further still another preferred embodiment of the present invention.

### DETAILED DESCRIPTION OF THE PREFERRED EMBODIMENTS

Referring now to the drawings, some preferred embodiments of the present invention will be described in detail.

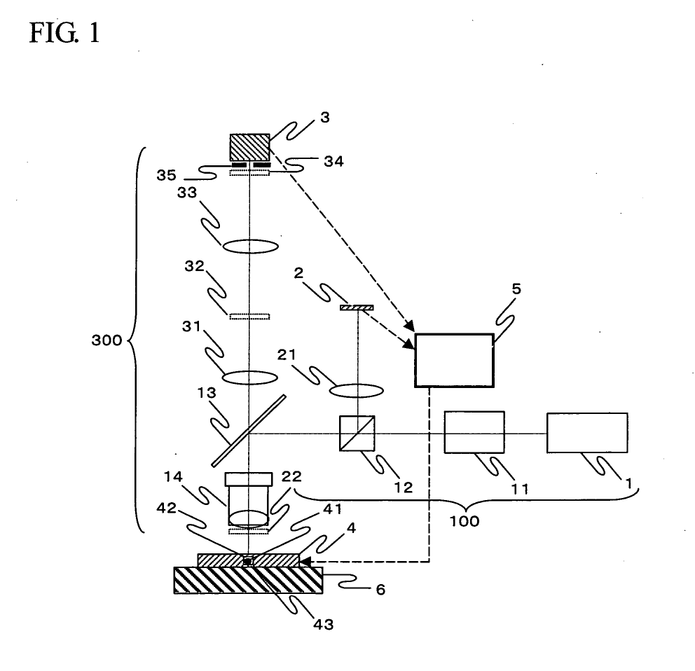

FIG. 1 shows one preferred embodiment of the present invention. An excitation light source 1 is an excitation light source for use in detecting fluorescence. Although this is usually a laser light source where a beam having a high directivity can be attained, an electrical discharging type light source may also be applicable. A laser beam forming optical system 11 radiates an optical beam emitted from the light source 1 under a desired shape against beads 41 acting as an inspected item or a measured item to be described later. When the excitation light attained by the light source 1 is a linear polarized light, it is preferable to use a polarized beam splitter as a beam splitter 12. The light passed through either the beam splitter or the polarized beam splitter is incident to a wavelength selection beam splitter 13. The wavelength selection beam splitter 13 reflects the excitation light and causes a fluorescence to pass through the splitter. After passing through an objective lens 14, the excitation light radiates against fluorescent substances on or in a plurality of beads 41 (dummy beads are designated by 42).

The fluorescent substances radiated with the excitation light emitted through the aforesaid radiating optical system 100 generate fluorescent light. The generated fluorescent light passes through the objective lens 14, shows a transmission through the wavelength selection beam splitter 13 and then the generated fluorescent light is detected by the fluorescent light detector 3 in a system 300 composed of units for guiding the fluorescent light to the fluorescent light detector 3. A detected fluorescent signal S_{L} is transmitted to a signal processing circuit 5 and an intensity of fluorescent light is detected accurately by a method to be described later.

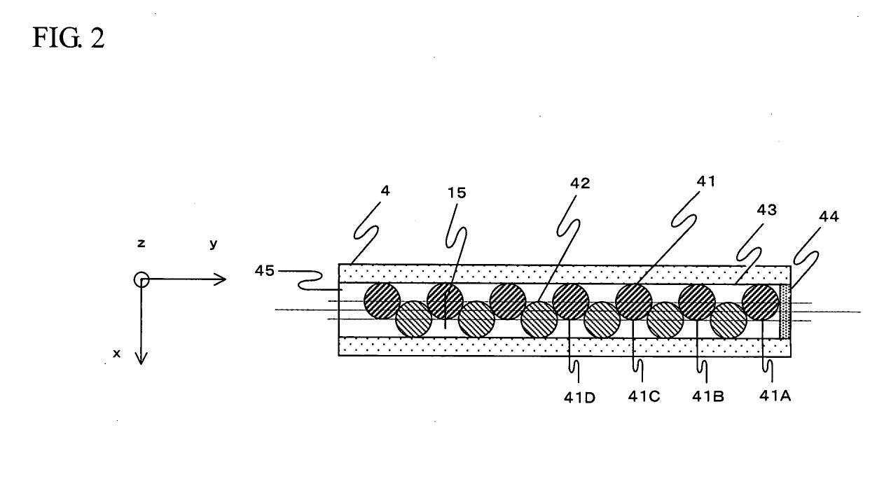

In FIG. 2 is illustrated a state of arrangement of the fluorescent beads in the flow path. In this preferred embodiment, the beads 41 of which surfaces have already been processed and non-processed usual beads 42 are arranged alternatively in such a way that the targets to be inspected such as DNA, protein or antibodies added with fluorescent substances are coupled to each other. As a result, the liquid becoming the target to be inspected such as DNA, protein or antibodies added with fluorescent substances flows along the flow path flowing from the left side to the right side as seen in FIG. 2, the liquid forms a large stream in a y-direction while becoming a disturbed flow near the beads, resulting in that the DNA, for example, of the aforesaid inspected item is efficiently hybridized on the surface processed beads.

The processed beads 41 that may be connected to the inspected items will be further processed such that the substances to be connected for every type of several kinds of DNA, protein or antibodies or the like are adhered to the beads surfaces in compliance with each of the items. Only at least the number of beads processed in this way are prepared and the beads are practically installed in such a way that they may be arranged in the flow path shown in FIG. 2 under a predetermined order, i.e. 41A, 41B, 41C and 41D . . . . A location of the end bead 41A is fixed with a stopper 44 having several small holes made to cause liquid to be passed.

Additionally, in order to increase a signal-to-noise ratio for detection of the adjoining inspected beads, some dummy beads 42 are arranged to hold the inspected beads. An excitation light emitted from the light source 1 such as an excitation light 15 shown in FIG. 2 passes through the beam forming means 11 and the objective lens 14 to radiate onto the beads in such a shape as one in which it is long in an x-direction perpendicular to a large flow y-direction in the flow path.

A stage 6 shown in FIG. 1 that can be moved mounts a sample having some beads arranged in the flow path and the stage can be moved in at least y-direction through a mechanism, not shown. Since the stage receives an instruction issued by a control circuit 5 to move in beads arrangement direction, the excitation light 15 scans the arranged beads in sequence and radiates thereto.

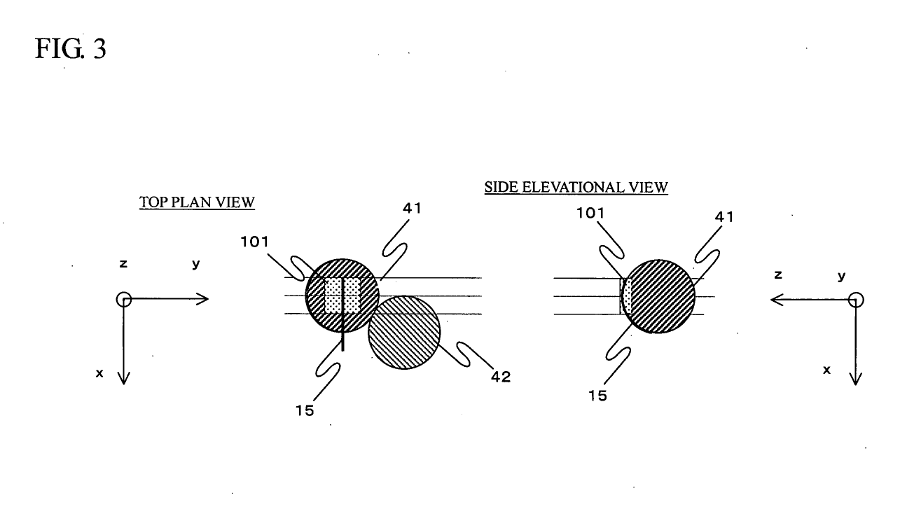

FIG. 3 is an enlarged view for showing the bead and the excitation light 15 for use in radiating against the bead. They are adjusted such that a center of the excitation light in its longitudinal direction and a center of the groove may coincide to each other. A width W of the groove and a bead diameter D have a following relation of:
W/2<D≦W
and they are arranged such that an order of the beads may not be disturbed.

In addition, it is desirable that a length L of the excitation light in its longitudinal direction has a following relation so as to cause the locations near their top portions may always be radiated even if the beads are arranged in a zigzag form. That is, detection of an area 101 on the bead shown in FIG. 3 reduces interference with a detection of the adjoining beads and causes a sufficient detection area to be assured. In FIG. 3, although the dummy beads are arranged alternately, when the dummy beads are not present, but when there are provided all the meaningful beads contributing to the detection, it becomes important to arrange the detection area shown in FIG. 3 for eliminating any influence of interference of the adjoining beads.

It is assumed that a length of the detection area 101 in a scanning direction in FIG. 3 is designated by Ly and a length of the excitation light in its longitudinal direction is designated by Lx. The value Ly will be determined by a method to be described later. In this case, the value Lx will be described. The value Lx is less than the width W of the groove. Even if the length is made to be longer than this value, fluorescence detection intensity becomes relatively low, resulting in that this variation provides no meaning at all. To the contrary, when the width W of the groove is set to be substantially equal to the bead diameter D, it would be satisfactory to set a width more than ⅓ of the bead diameter. Setting the value Lx to an excessive low value under a condition in which the groove width W is larger than the bead diameter D causes the excitation light not to enable the center of the bead to be radiated and a detection precision is reduced.

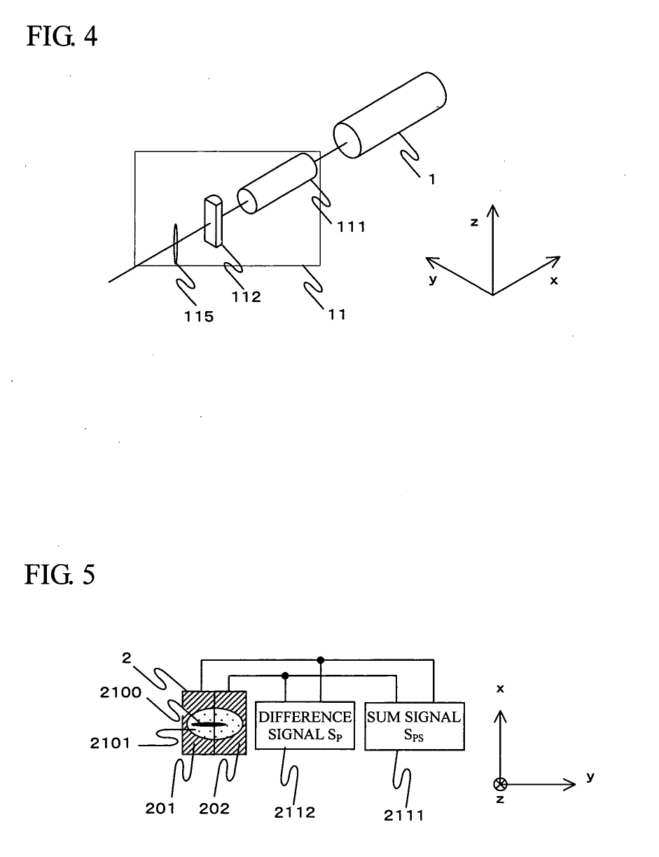

In FIG. 4 is illustrated a configuration of the beam forming optical system 11 shown in FIG. 1. In FIG. 4, a laser beam emitted from the laser light source 1 has a diameter of about 1 mm, so that a beam diameter is expanded from several millimeters to several tens of millimeters through a beam expander 111. The expanded beam shows that a longitudinal direction of a spot near a focal point 115 of a cylindrical lens 112 keeps a length ranging from several millimeters to several tens of millimeters and its narrow direction is narrowed to about sub-millimeters through the cylindrical lens. This focal point position occupies a substantial conjugate positional relation with the top point of the inspected bead through the objective lens. Accordingly, the laser beam passed through the beam forming optical system 11 radiates a fine excitation light elongated in a direction perpendicular to a direction of groove onto the beads.

The excitation light passed through the beam forming optical system is absorbed by the fluorescent substance added to the beads to generate fluorescence. The generated fluorescence passes through the objective lens, passes through the wavelength selection beam splitter and the fluorescence is detected by a high sensitivity light detector 3 through lenses 31, 33. The high sensitivity light detector is set such that a photo-diode and a photo-multi-processor and the like are selected according to an intensity of the detected light intensity. The front surface of the high sensitivity light detector is provided with a slit aperture at a location where it is conjugate with surfaces of the beads so as to cause only a fluorescence emitted from the location where excitation light radiates to be detected.

The interference filter 34 shown in FIG. 1 is used when a sufficient removal of excitation light cannot be attained only through the wavelength selection beam splitter. Almost of all the excitation light do not reach to the high sensitivity light detector due to this filter. Further, in order to eliminate either a reflection of the excitation light from the groove bottom part of the bead samples or the lid surface of the groove or some optical parts such as lenses in the midway part of the light path and fluorescence, there is sometimes provided a noise elimination spatial filter for use in eliminating these noises.

The excitation light for use in radiating against the beads in its fine elongated form returns back to its light path from which the excitation light came. Accurate detection of the location where the beads are radiated with the light could be carried out by applying the method described below. That is, the returning excitation light reflected by the wavelength selection beam splitter shown in FIG. 1 is curved upwardly in its light path by the beam splitter 12, passes through the lens 21 and then the light is received by the position sensor 2.

FIG. 5 illustrates architecture of the position sensor 2. The position sensor 2 is separated into two detector elements 201, 202 at its central part and then a light intensity detected at each of the right and left light receiving surfaces is independently calculated. The light 2100 having the excitation light normally reflected at the bead surfaces and directly incident to the position sensor and the light 2101 or the like reflected at the location other than that of the beads are incident to the receiving surfaces. As shown in FIG. 1 or FIGS. 2, 3, although the light 2100 normally reflected at the beads and returned back is a fine illuminating light elongated in an x direction, it is elongated in the y direction on the position sensor because the position sensor and the bead surfaces have a Fourier conversion relation. The two elements 201, 202 of the position sensor are arranged in this longitudinal direction.

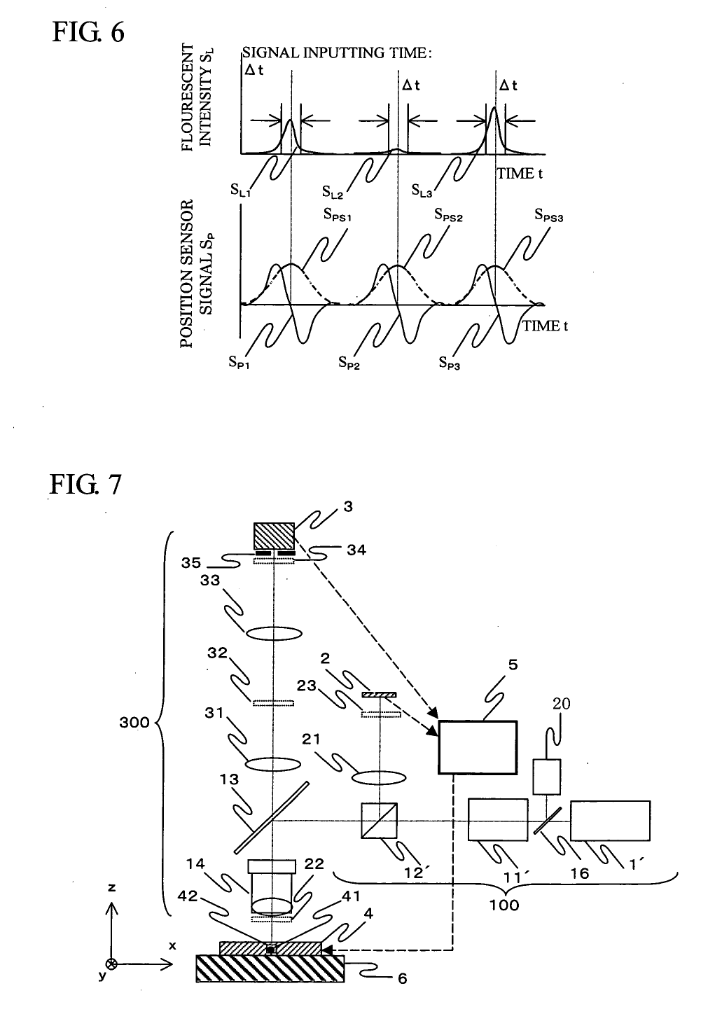

When the fine elongated excitation light radiates against the top parts of the beads, i.e. their centers, the excitation light normally reflected with the beads is set such that a center of the distribution of intensity comes to an intermediate position between the two elements 201, 202 of the position sensor. When the excitation light is displaced from the top portions of the beads to the direction of +y and radiated, the light is shifted toward the two elements 201 of the position sensor and received, resulting in that when the light is displaced in −y direction, the light is shifted to the element 202. With such a configuration as above, as shown in FIG. 6, a difference signal Sp and a sum signal S_{PS} can be attained through a relative scanning of the excitation light and the beads as shown in a lower graph in FIG. 6 through a circuit for attaining the difference signal Sp and the sum signal S_{PS} in reference to the detected signals of the two elements 201, 202.

A waveform shown at the lower graph of FIG. 6 is generated. The excitation light is scanned in a relative manner, and a time in which the light passes near the center of the beads as described below can be made apparent in reference to the difference signals S_{P1}, S_{P2} and S_{P3} and the sum signals S_{PS1}, S_{PS2} and S_{PS3}. That is, a timing in which the signals S_{PS1}, S_{PS2} and S_{PS3} are more than a certain level and the signals S_{P1}, S_{P2} and S_{P3} become 0 is detected by a processing circuit. Fluorescence detection intensities between time widths Δt before and after this time are multiplied.

Since a fluorescence detection intensity is detected in a waveform as indicated by the upper graph in FIG. 6, the fluorescent signals S_{L1}, S_{L2} and S_{L3} are detected by the high intensity light detector 3 at a timing having the highest fluorescent detection between the times Δt, i.e. at a location where the excitation light radiates against a part near the upper portions of the beads, resulting in that a quite accurate fluorescent detection can always be carried out. The fluorescent intensity of each of the beads can be obtained when the fluorescent detection signals S_{L} are accumulated in sequence in a memory, a plurality of data corresponding to the time widths Δt determined mainly based on an address in the memory when the center of the beads is detected are taken out and their sums are taken.

In the foregoing description, although there has been described that the beam splitter shown in FIG. 1 is applied as a usual beam splitter, some advantages using a polarized beam splitter will be described as a preferred embodiment. The polarized beam splitter 12 is set such that a polarizing direction of the linear polarization of the laser light source 1 is a z direction. Since the laser beam of the linear polarization is incident to the polarized beam splitter 12 as a P polarization, the beam may pass through it by approximately 100%. After the linear polarization of the passed S-polarization passes through the objective lens 14, it passes through ¼ wavelength plate 22 to become a circular polarization, radiates against the beads and when the reflected light passes again through the ¼ wavelength plate 22 from an opposite direction, it is changed into an S-polarization and returns back to the polarization beam splitter 12. The linear polarization of this S-polarization is reflected by the polarization beam splitter by approximately 100% and reaches to the position sensor 2.

With such an optical system having the aforesaid configuration, all the light reflected at the surface of the optical system present between the polarization beam splitter and the ¼ wavelength plate and returned back to the position sensor as noise pass through the polarized beam splitter and do not reach to the position sensor. Due to this fact, light becoming noise does not enter into the position sensor and it becomes possible to detect accurately a time in which the excitation light passes through the center of each of the beads and further a high precise detection of fluorescent can be carried out.

FIG. 7 illustrates another preferred embodiment of the present invention. The component elements in FIG. 7 having the same reference numbers as those shown in FIG. 1 denote the same component elements. In this preferred embodiment, as a light source 1′, an electrical discharging lamp such as a mercury lamp is used. In the beam forming optical system 11′, a fine elongated sight view diaphragm (115 in FIG. 4) is inserted into a position in conjugate with a position of each of the beads in addition to the cylindrical lens for use in forming a fine elongated beam. A laser light source 20 for accurately setting a center of the bead is separately arranged because a directivity of the excitation light cannot be attained like that of the laser beam. The light emitted from this laser light source is weak as compared with that of the excitation light, and its wavelength is long. In addition, its wavelength is a long wavelength different from a fluorescence wavelength in the way that it may not become a noise at the time of fluorescent detection. With such an arrangement as above, even if a directivity of the excitation light is not well, the center of the bead can be set accurately and the fluorescence of the bead can be accurately detected in the same manner as that in the preferred embodiment described above in reference to FIG. 1.

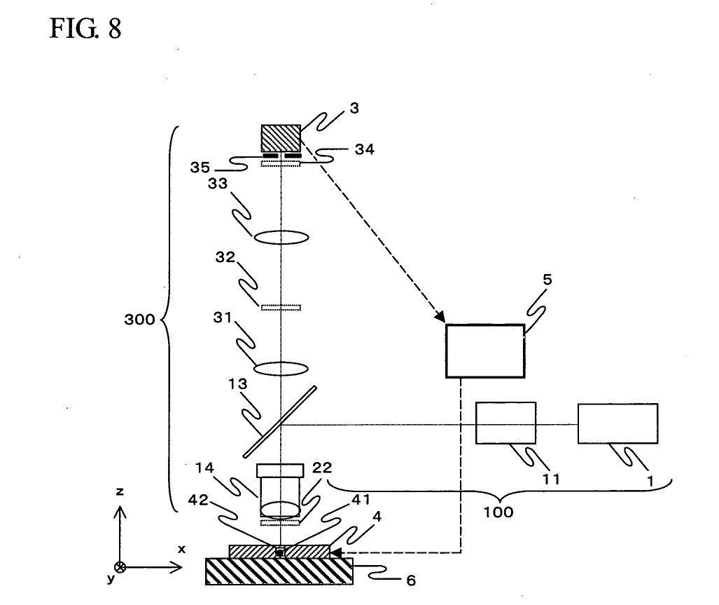

FIG. 8 shows a still another preferred embodiment of the present invention. The same reference numbers as those shown in FIG. 1 denote the same component elements. In this preferred embodiment, although a shape of the excitation light on the bead is not different from that illustrated in the aforesaid preferred embodiment, a position of the regular reflected light is not carried out. That is, a detection timing for the bead is determined under application of only the detecting signal of the high sensitivity light detector 3. When the number of beads is less or the arrangement of the beads is in a regular form or the fluorescence more than a certain degree can be detected from the beads and the like, the detection can be attained only through application of the fluorescence detection signal.

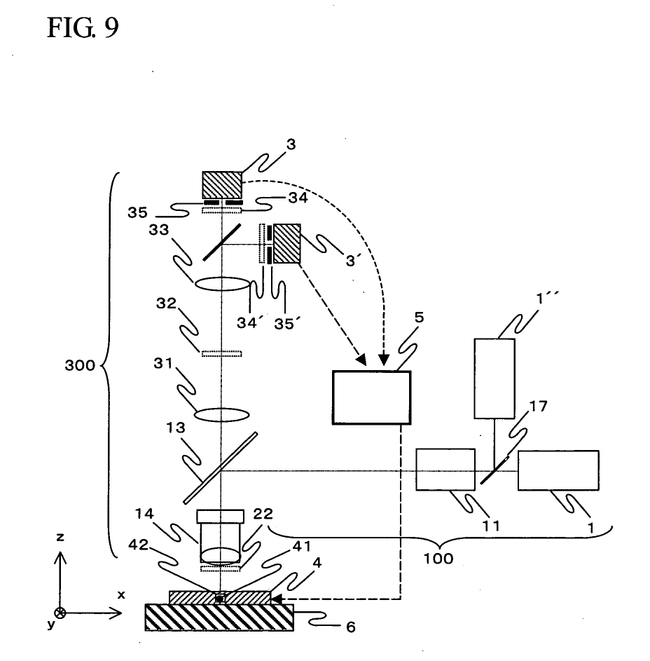

FIG. 9 shows a still further preferred embodiment of the present invention. In this preferred embodiment, as the excitation light, the excitation lights having more than two types of wavelength are applied. The light source 1 is a light source with a wavelength of 635 nm and the light source 1′ is a light source with a wavelength of 532 nm. A wavelength selection splitter 17 synthesizes these lights having two different wavelengths. The beam forming optical system 11 causes both lights to be changed into a fine elongated shape on the beads. Since the beads are added with more than two kinds of fluorescence, each of the fluorescent substances may absorb much amount of excitation light with the corresponding excitation light to emit fluorescence. Accordingly, the produced fluorescence is reflected and passed according to each fluorescence by the wavelength selection beam splitter 36 and detected by the high sensitivity light detectors 3, 3′ corresponding to each of the fluorescence, thereby a plurality of fluorescence can be separately detected. It is naturally to say in this case that the most-preferable interference filters 35, 35′ are applied in compliance with a wavelength of each of the fluorescence.

According to the present invention, it becomes possible to detect fluorescence easily and accurately against the sample having some beads added with fluorescent substances, becomes possible to perform a convenient and less-expensive inspection and so the beads may become an effective tool for a user participated in research and development in the filed of a life science. It is certain that the present invention may contribute to a development in the field of the life science and a development in medical diagnosis or medicines and the like.

1. A fluorescence beads detecting method for radiating an excitation light to beads including on or in fluorescent substances arranged in a flow path to detect fluorescence emitted from the beads, wherein the excitation light radiated to the beads has a shape having different horizontal to vertical ratio, a longitudinal direction of the shape is directed in a direction perpendicular to a larger flow in a flow path as compared with a short direction, positions on the beads to which the excitation light radiates are relatively changed in sequence, the fluorescence generated through radiation of the excitation light is detected, being separated from the excitation light and a desired fluorescent signal is attained.

2. The fluorescent beads detecting method according to claim 1, wherein a length of the excitation light in the long direction is smaller than a diameter of section perpendicular to a flow direction in the flow path and 0.3 times larger than the diameter of the bead.

3. The fluorescent beads detecting method according to claim 1, wherein light reflected at the beads by either the excitation light or a position detecting beam keeping a certain positional relation with the excitation light is guided to a light position detector, a fluorescence detection timing is determined based on a position signal detected by the light position detector and the fluorescence is detected.

4. The fluorescent beads detecting method according to claim 1, wherein when fluorescence generated through radiation of the excitation light is detected, being separated from the excitation light, at least one type of excitation light-fluorescence separating means is applied from among three units of a wavelength selection beam splitter, a spatial filter and an interference filter.

5. The fluorescent beads detecting method according to claim 1, wherein either the excitation light or the position detecting beam causes light linearly polarized through application of a polarizer to either a light source of linear polarized light or a light source of non-polarized light to be incident to the light position detector by applying the polarized beam splitter and the wavelength plate in a state where the light reflected by the beads has a superior efficiency and has a less amount of noise light.

6. The fluorescent beads detecting method according to claim 5, wherein the wavelength plate is a ¼ wavelength plate and is arranged between an optical system facing against the beads and the beads.

7. The fluorescent beads detecting method according to claim 1, wherein there is provided a sample in which the fluorescent beads including the fluorescent substances on or in the beads, dummy beads not including any fluorescent substances on or in the beads and the fluorescent beads including different kinds of fluorescent substances are arranged in a certain order.

8. A fluorescent beads detecting apparatus for radiating an excitation light to beads including on or in the beads fluorescent substances arranged in a flow path and detecting the fluorescence emitted from the beads, comprising:
an exciting light source;
a beam forming optical system for forming light beams emitted from the light source in a shape having a different horizontal to vertical ratio on the fluorescent beads and with a long direction of the shape being directed toward a direction perpendicular to a large flow in the flow path as compared with that of the short direction;
an excitation light-fluorescent branch means for separating the excitation light from the fluorescence;
a wavelength selection beam splitter for either reflecting or passing light depending on a difference in wavelength between the excitation light and the fluorescence;
a radiation optical system for radiating (excitation light) the excitation light passed through the beam splitter to the beads including at the surfaces or inner portions the fluorescent substance acting as detected item on or in the beads;
a detecting optical system for detecting the fluorescence emitted from the fluorescent substance;
a fluorescent detector for detecting fluorescence passed through the detecting optical system;
a position detector to which an excitation light reflected from the beads or light emitted from the light source separately arranged from the excitation light is radiated so as to detect the light reflected from the beads and passed through the excitation light beam splitter;
a fluorescent detector; and
a control circuit for attaining information of the fluorescent beads based on information obtained from the position detector and the fluorescent detector.

9. The fluorescent detecting apparatus according to claim 8, wherein the lighting optical system and the detecting optical system are a common objective lens.

10. The fluorescent detecting apparatus according to claim 9, wherein the beam splitter is a polarized beam splitter, and a ¼ wavelength plate is installed between the objective lens and the beads.

11. The fluorescent detecting apparatus according to claim 8, wherein at least one type of excitation light-fluorescent separating means of the excitation light-fluorescent separating means from among three units of the wavelength selection beam splitter, spatial filter and interference filter as the excitation light-fluorescent branch means.

12. The fluorescent detecting apparatus according to claim 8, wherein there is provided a driving means for displacing a relative position between the fluorescent beads and the excitation light.
